(19) United States
(12) Patent Application Publication
(10) Pub. No.: US 2010/0119726 A1
(43) Pub. Date: May 13, 2010

(54) Group 2 Metal Precursors For Deposition Of Group 2 Metal Oxide Films

(75) Inventors: Xinjian Lei, Vista, CA (US); Daniel P. Spence, Carlsbad, CA (US); John Anthony Thomas Norman, Encinitas, CA (US); Michael Ulman, Mertztown, PA (US)

Correspondence Address: AIR PRODUCTS AND CHEMICALS, INC.;PATENT DEPARTMENT, 7201 HAMILTON BOULEVARD, ALLENTOWN, PA, 181951501, US

(73) Assignee: Air Products and Chemicals, Inc., Allentown, PA (US)

(21) Appl. No.: 12/266,806

(22) Filed: Nov. 7, 2008

(51) Int. Cl. C07C 249/00; C07D 307/08; C09D 4/00; B05D 5/12; C23C 4/10

(52) U.S. Cl. 427/453; 549/491; 564/248; 106/287.2; 106/287.25; 427/126.3

(57) ABSTRACT

This invention is related to Group 2 metal-containing polydentate β-ketoiminate precursors and compositions comprising Group 2 metal-containing polydentate β-ketoiminate precursors, wherein the polydentate β-ketoiminate precursors incorporate an alkoxy group in the imino portion of the molecule. The compounds and compositions are useful for fabricating metal containing films on substrates such as silicon, metal nitride, metal oxide and other metal layers via chemical vapor deposition (CVD) processes.

### BACKGROUND OF THE INVENTION

The present invention generally relates to Group 2 metal-organic complexes and their liquid compositions which are suitable for use to deposit a layer of the Group 2 metal-containing oxide thereof in chemical vapor deposition processes such as, for example, cyclic chemical vapor deposition (CCVD) or atomic layer deposition (ALD) on a semi-fabricated semiconductor substrate. More particularly, the Group 2 metal-organic complexes comprise polydentate β-ketoiminate ligands that are organic in character and are stable at ambient conditions.

In the semiconductor industry there is a growing need for volatile sources of different metal precursors to be used in the chemical vapor deposition (CVD) of Group 2 alkaline earth metal-containing oxide films and the like. The key property required for such metal sources is that they readily evaporate or sublime to give a metal containing vapor which can be decomposed in a controlled manner to deposit a film onto a target substrate. In this application, as well as for other applications, it is desirable that the alkaline earth compounds have a high volatility, which facilitates sublimation and the transport to the place of deposition, that they can deposit films thermally with or without oxidizing agents at relatively low temperatures, such as those of the order of 450° C., and that, for certain application areas, they are deposited in the form of the oxides and not, for example, in the form of other decomposition products such as carbonates.

Alkaline earth metal β-diketoiminates and certain derivatives have already been used for the CVD method. Complexes of alkaline earth metals and multidentate ligands have been employed in the prior art as precursors, however, such complexes are often present as oligomers whose volatility and stability are often unsatisfactory. Accordingly, the semiconductor fabrication industry continues to demand novel metal sources containing precursors for CVD processes including atomic layer deposition (“ALD”) for fabricating conformal metal containing films on substrates such as silicon, metal nitride, metal oxide and other metal-containing layers using these metal-containing precursors.

### BRIEF SUMMARY OF THE INVENTION

In one aspect, the present invention provides a polydentate β-ketoiminate selected from the group consisting of:

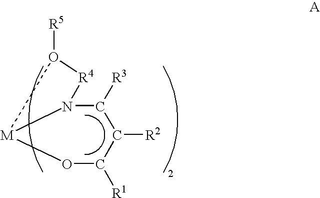

wherein M is a Group 2 metal selected from the group consisting of: magnesium, calcium, strontium, and barium; R¹ is selected from the group consisting of: a C₁ to C₁₀ alkyl, a C₁ to C₁₀ alkoxyalkyl, a C₁ to C₁₀ alkoxy, a C₁ to C₁₀ fluoroalkyl, a C₃ to C₁₀ cycloaliphatic, and a C₆ to C₁₀ aryl; R² is selected from the group consisting of: hydrogen, a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl; R³ is selected from the group consisting of: a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl; R⁴ is a C₁ to C₆ linear or branched alkylene; and R⁵ is selected from the group consisting of: a C₁ to C₁₀ alkyl, a C₁ to C₁₀ fluoroalkyl, a C₃ to C₁₀ cycloaliphatic, and a C₆ to C₁₀ aryl,

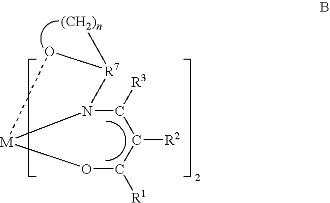

wherein M is a Group 2 metal selected from the group consisting of: magnesium, calcium, strontium, and barium; R¹ is selected from the group consisting of: a C₁ to C₁₀ alkyl, a C₁ to C₁₀ alkoxyalkyl, a C₁ to C₁₀ alkoxy, a C₁ to C₁₀ fluoroalkyl, a C₃ to C₁₀ cycloaliphatic, and a C₆ to C₁₀ aryl; R² is selected from the group consisting of: hydrogen, a C₁ to C₁₀ alkyl, a C₁ to C₁₀ alkoxyalkyl, a C₃ to C₁₀ cycloaliphatic, and a C₆ to C₁₀ aryl; R³ is selected from the group consisting of: a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl; R⁷ is an organic group comprising 2 or 3 carbon atoms, thus making a five- or six-member coordinating ring to the metal center; and n=3, 4, 5, and

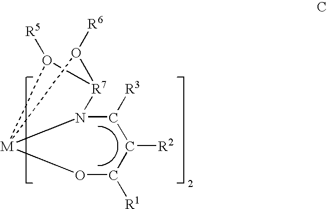

wherein M is a Group 2 metal selected from the group consisting of: magnesium, calcium, strontium, and barium; R¹ is selected from the group consisting of: a C₁ to C₁₀ alkyl, a C₁ to C₁₀ alkoxyalkyl, a C₁ to C₁₀ alkoxy, a C₁ to C₁ fluoroalkyl, a C₃ to C₁₀ cycloaliphatic, and a C₆ to C₁₀ aryl; R² is selected from the group consisting of: hydrogen, a C₁ to C₁₀ alkyl, a C₁ to C₁₀ alkoxyalkyl, a C₃ to C₁₀ cycloaliphatic, and a C₆ to C₁₀ aryl; R³ is selected from the group consisting of: a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl; R⁷ an organic group comprising 2 or 3 carbon atoms; and R⁵ and R⁶ are each independently selected from the group consisting of: a C₁ to C₁₀ alkyl, a C₁ to C₁₀ fluoroalkyl, a C₃ to C₁₀ cycloaliphatic, and a C₆ to C₁₀ aryl.

In another aspect, the present invention provides a polydentate β-ketoiminate as defined above that is free of fluoroalkyl groups.

In yet another aspect, the present invention provides a liquid composition suitable for use to deposit a Group 2 metal-containing film on a substrate by a chemical vapor deposition process or an atomic layer deposition, the composition comprising: a polydentate β-ketoiminate according to the present invention; and a solvent selected from the group consisting of: aliphatic hydrocarbons, aromatic hydrocarbons, ethers, esters, nitrites, organic esters; organic amines, polyamines, organic amides, alcohols, and mixtures thereof.

### BRIEF DESCRIPTION OF SEVERAL VIEWS OF THE DRAWINGS

FIG. 1 is a crystal structure of bis(2,2-dimethyl-5-[(2-methoxyethyl)amino]hex-4-en-3-onato)strontium;

FIG. 2 is a TGA of bis(2,2-dimethyl-5-[(2-methoxyethyl)amino]hex-4-en-3-onato)strontium (solid line) vs. 2,2-dimethyl-5-[(2-methoxyethyl)amino]hex-4-en-3-one (dashed line);

FIG. 3 is a crystal structure of bis(2,2-dimethyl-5-[(2-methoxy-1-methylethyl)amino]hex-4-en-3-onato)strontium;

FIG. 4 is a TGA of bis(2,2-dimethyl-5-[(2-methoxy-1-methylethyl)amino]hex-4-en-3-onato)strontium; and

FIG. 5 is a crystal structure of bis(2,2-dimethyl-5-[(2,2-dimethoxyethyl)amino]hex-4-en-3-onato)barium.

### DETAILED DESCRIPTION OF THE INVENTION

This invention is related to Group 2 metal-containing polydentate β-ketoiminate precursors and compositions comprising Group 2 metal-containing polydentate β-ketoiminate precursors, wherein the polydentate β-ketoiminate precursors incorporate an alkoxy group in the imino portion of the molecule. The compounds and compositions are useful for fabricating metal containing films on substrates such as silicon, metal nitride, metal oxide and other metal layers via chemical vapor deposition (CVD) processes. As used herein, the term “chemical vapor deposition processes” refers to any process wherein a substrate is exposed to one or more volatile precursors, which react and/or decompose on the substrate surface to produce the desired deposition. Examples include plasma enhanced chemical vapor deposition (PECVD), low pressure chemical vapor deposition (LPCVD), direct liquid injection chemical vapor deposition (DLCVD), hot wire chemical vapor deposition (HWCVD), cyclic chemical vapor deposition (CCVD), molecular layer deposition (MLD), atomic layer deposition (ALD), and metal-organic chemical vapor deposition (MOCVD). The deposited metal films (which includes Group 2 metal oxide films) have applications ranging from computer chips, optical device, magnetic information storage, to metallic catalyst coated on a supporting material.

The metal-containing multidentate β-ketoiminate precursors of the present invention comprise Group 2 metals of the Periodic Table of Elements. The Group 2 metals include beryllium (Be), magnesium (Mg), calcium (Ca), strontium (Sr), barium (Ba) and radium (Ra). In preferred embodiments of the present invention, the Group II metal is calcium, strontium, or barium. In more preferred embodiments of the present invention, the Group 2 metal is strontium or barium.

The multidentate polydentate β-ketoiminates according to the present invention preferably incorporate an alkoxy group in the imino group. The multidentate polydentate β-ketoiminates are selected from the group represented by the Structures A, B, and C. Structure A is defined as

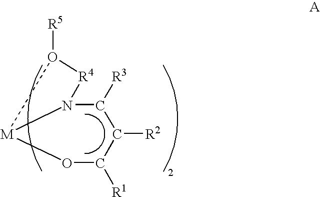

wherein M is a Group 2 metal such as, for example, magnesium, calcium, strontium, and barium. Preferably, M is strontium or barium. The organo groups (i.e., the R groups) employed in the complexes of the present invention may include a variety of organo groups and they may be linear or branched. In preferred embodiments, R¹ is selected from the group consisting of: a C₁ to C₁₀ alkyl, a C₁ to C₁₀ alkoxyalkyl, a C₁ to C₁₀ alkoxy, a C₁ to C₁₀ fluoroalkyl, a C₁ to C₁₀ cycloaliphatic, and a C₆ to C₁₀ aryl. As used herein, the group “alkoxyalkyl” refers to an ether-like moiety that includes a C—O—C fragment. Examples include —CH₂CH₂—O—CH₂CH₂—O—CH₃ and —CH₂CH₂—O—CH₂—O—CH₃. Preferably, R¹ is a bulky alkyl group containing 4 to 6 carbon atoms such as, for example, a tert-butyl group, a sec-butyl, and a tert-pentyl group. The most preferred R¹ group is tert-butyl or tert-pentyl. Preferably, R² is selected from the group consisting of: hydrogen, a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl. More preferably, R² is hydrogen, or a C₁ to C₂ alkyl. Preferably, R³ is selected from the group consisting of: a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl. More preferably, R³ is a C₁ to C₂ alkyl. Preferably, R⁴ is a C₁ to C₆ linear or branched alkylene and, more preferably, R⁴ contains a branched alkylene bridge containing 3 or 4 carbon atoms and having at least one chiral center carbon atom. Without intending to be bound by a particular theory, it is believed that the chiral center in the ligand plays a role in lowering the melting point as well as increasing the thermal stability of the complex. Preferably, R⁵ is selected from the group consisting of: a C₁ to C₁₀ alkyl, a C₁ to C₁₀ fluoroalkyl, a C₃ to C₁₀ cycloaliphatic, and a C₆ to C₁₀ aryl. More preferably, R⁵ is a C₁ to C₂ alkyl.

Structure B is defined as

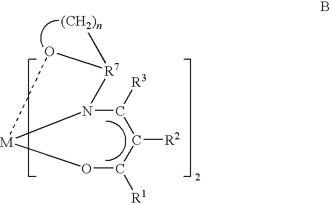

wherein M is a Group 2 metal selected from magnesium, calcium, strontium, barium, and n is 4, 5, or 6. Preferably, M is strontium or barium. The organo groups (i.e., the R groups) employed in the complexes of the present invention may include a variety of organo groups. In preferred embodiments, R¹ is selected from the group consisting of: a C₁ to C₁₀ alkyl, a C₁ to C₁₀ alkoxyalkyl, a C₁ to C₁₀ alkoxy, a C₁ to C₁₀ fluoroalkyl, a C₃ to C₁₀ cycloaliphatic, and a C₆ to C₁₀ aryl. Preferably, R¹ is a bulky alkyl group containing 4 to 6 carbon atoms such as, for example, a tert-butyl group, a sec-butyl group, and a tert-pentyl group. The most preferred R¹ group is tert-butyl or tert-pentyl. Preferably, R² is selected from the group consisting of: hydrogen, a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl, more preferably, R² is hydrogen, a C₁ to C₂ alkyl. Preferably, R³ is selected from the group consisting of: a C₁ to C₁₀ alkyl, a C₁ to C₁₀ alkoxyalkyl, a C₁ to C₁₀ alkoxy, a C₃ to C₁₀ cycloaliphatic and a C₆ to C₁₀ aryl. More preferably, R³ is a C₁ to C₂ alkyl. Preferably, R⁷ is a C₁ to C₆ linear or branched alkylene and, more preferably, R⁷ is an organic group comprising 2 or 3 carbon atoms, thus making a five- or six-member coordinating ring to the metal center, and n=3, 4, or 5. In preferred embodiments, R⁷ is

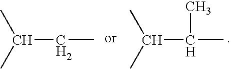

Structure C is defined as

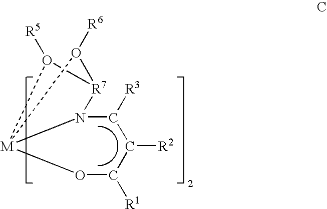

wherein M is a Group 2 metal selected from magnesium, calcium, strontium, and barium. Preferably, M is strontium or barium. The organo groups (i.e., the R groups) employed in the complexes of the present invention may include a variety of organo groups. In preferred embodiments, R¹ is selected from the group consisting of: a C₁ to C₁₀ alkyl, a C₁ to C₁₀ alkoxyalkyl, a C₁ to C₁₀ alkoxy, a C₁ to C₁₀ fluoroalkyl, a C₃ to C₁₀ cycloaliphatic, and a C₆ to C₁₀ aryl. Preferably, R¹ is a bulky alkyl group containing 4 to 6 carbon atoms such as, for example, a tert-butyl group, sec-butyl group, and a tert-pentyl group. The most preferred R¹ group is tert-butyl or tert-pentyl. Preferably, R² is selected from the group consisting of: hydrogen, a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl. More preferably, R² is hydrogen or a C₁ to C₂ alkyl. Preferably, R³ is selected from the group consisting of: hydrogen, a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl. More preferably, R³ is a C₁ to C₂ alkyl. Preferably, R⁷ is selected from the group consisting of: a C₂ to C₆ linear or branched alkylene, a C₁ to C₁₀ fluoroalkylene, and a C₆ to C₁₀ aryl and, more preferably, R⁷ is an organic group comprising 2 or 3 carbon atoms, thus making a five- or six-member coordinating ring to the metal center. In preferred embodiments, R⁷ is

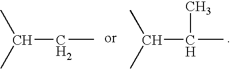

Preferably, R⁵ and R⁶ are selected from the group consisting of: a C₁ to C₁₀ alkyl, a C₁ to C₁₀ fluoroalkyl, a C₃ to C₁₀ cycloaliphatic, and a C₁ to C₁₀ aryl.

In preferred embodiments of the present invention, the compounds of Structures A, B, and C as defined above are free of fluoroalkyl groups.

The polydentate β-ketoiminate ligands according to the present invention can be prepared by well known procedures such as the Claisen condensation of a bulky ketone and an ethyl ester in presence of a strong base such as, for example, sodium amide or hydride to provide a diketone, followed by another known procedure such as Schiff base condensation reaction with alkoxyalkylamine. The Schiff base condensation reaction, for example, is shown below:

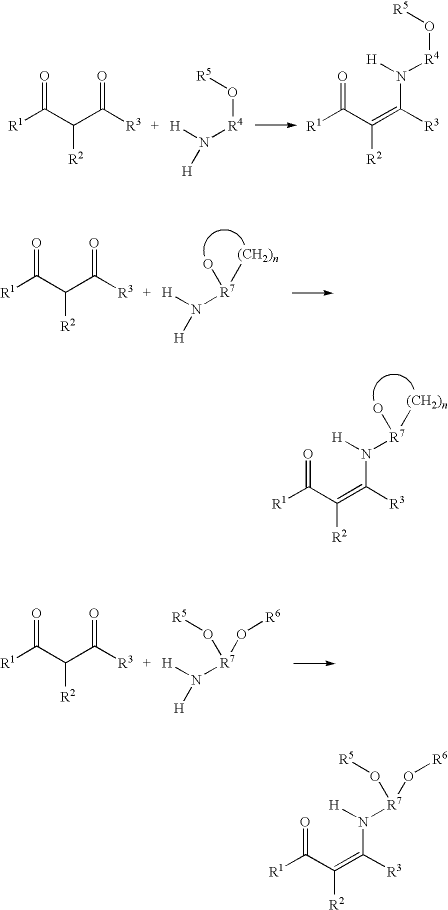

The resulting ligands can be purified via vacuum distillation for a liquid or crystallization for solid. As a discussed above, it is preferred to employ a bulky R¹ group, e.g., C₄₋₁₀ branched alkyl groups without hydrogen attached to the carbon connected to the ketone functionality because the bulky R¹ group prevents any side reactions occurring in the Schiff condensation and later protecting the metal centers from inter-molecular interaction. It should be noted, however, that the R¹⁻⁶ groups in the polydentate ligands should be as small as possible in order to decrease the molecular weight of the resulting metal-containing complexes which will allow the complexes to have a sufficiently high vapor pressure.

The metal-containing complexes of the present invention can then be prepared via the reaction of the multidentate ketoiminato ligands with metal alkoxide in a solvent as shown below:

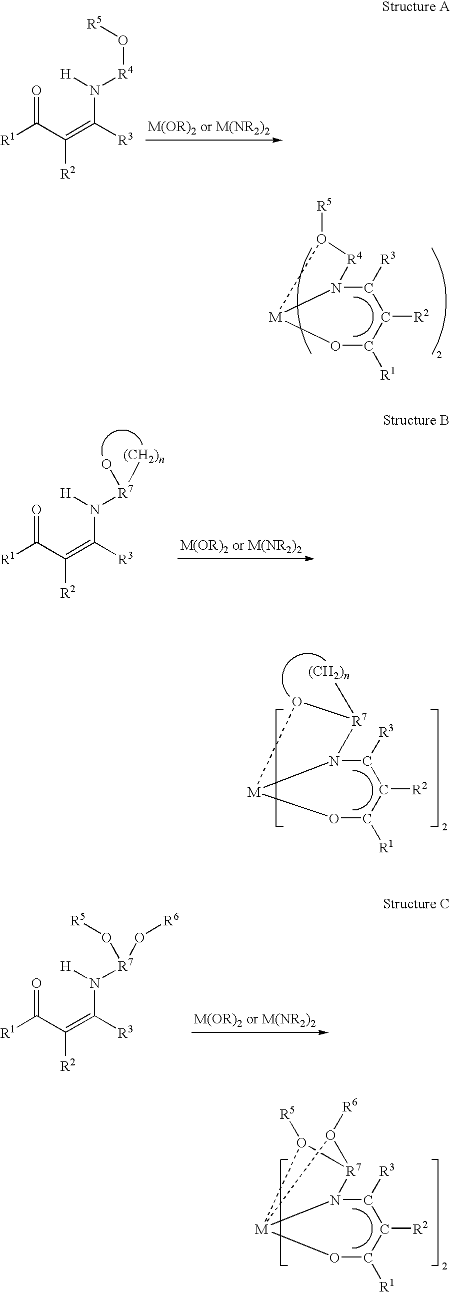

The metal, M, is as defined above. Preferably, the oxide portion of the metal alkoxide is selected form the group consisting of: methoxide, ethoxide, iso-propoxide, n-propoxide, tert-butoxide, sec-butoxide, iso-butoxide, and mixtures thereof. In preferred embodiments, the metal alkoxide is strontium iso-propoxide.

Suitable solvents for use in the reaction according to the present invention include, for example, THF, toluene, hexane, an ether, and mixtures thereof. THF is the preferred solvent. In preferred embodiments, the reaction according to the present invention occurs at a temperature of from about 10° C. to about 150° C. In preferred embodiments, the reaction occurs under ambient temperature or reflux at the boiling point of the solvent.

The compounds of each of Structures A, B, and C are solids at room temperature which can be purified via crystallization or sublimation.

Group 2 metal-containing complexes with multidentate β-ketoiminate ligands according to the present invention may be employed as precursors to make thin metal oxide films via either the CVD, CCVD or ALD techniques at temperatures less than 500° C. The CVD process can be carried out with or without oxidizing agents whereas an ALD or CCVD process usually involves the employment of another reactant such as an oxidizing agent.

Oxidizing agents for chemical vapor deposition or atomic layer deposition processes include oxygen, hydrogen peroxide and ozone and plasma oxygen. For multi-component metal oxides, the Group 2 metal-containing complexes according to the present invention can be premixed if they have the same multidentate β-ketoiminate ligands. These Group 2 metal-containing complexes with multidentate β-ketoiminate ligands can be delivered in vapor phase into, for example, a CVD or ALD reactor via well-known bubbling or vapor draw techniques. A direct liquid delivery method can also be employed by dissolving the complexes in a suitable solvent or a solvent mixture to prepare a solution with a molar concentration from 0.001 to 2 M depending the solvent or mixed-solvents employed.

The solvent employed in solubilizing the precursor for use in a deposition process may comprise any compatible solvent or their mixture including aliphatic hydrocarbons, aromatic hydrocarbons, ethers, esters, nitrites, and alcohols. The solvent component of the solution preferably comprises a solvent selected from the group consisting of glyme solvents having from 1 to 20 ethoxy —(C₂H₄O)— repeat units; C₂-C₁₂ alkanols, organic ethers selected from the group consisting of dialkyl ethers comprising C₁-C₆ alkyl moieties, C₄-C₈ cyclic ethers; C₁₂-C₆₀ crown O₄-O₂₀ ethers wherein the prefixed Cᵢ range is the number i of carbon atoms in the ether compound and the suffixed Oᵢ range is the number i of oxygen atoms in the ether compound; C₆-C₁₂ aliphatic hydrocarbons; C₆-C₁₈ aromatic hydrocarbons; organic esters; organic amines, polyamines and organic amides.

Another class of solvents that offers advantages is the organic amide class of the form RCONR′R″ wherein R and R′ are alkyl having from 1-10 carbon atoms and they can be connected to form a cyclic group (CH₂)ₙ, wherein n is from 4-6, preferably 5, and R″ is selected from alkyl having from 1 to 4 carbon atoms and cycloalkyl. N-methyl- or N-ethyl- or N-cyclohexyl-2-pyrrolidinones, N,N-Diethylacetamide, and N,N-Diethylformamide are examples.

In one embodiment of the method of the present invention, a cyclic deposition process such as CCVD, ALD, or PEALD may be employed, wherein a Group 2 metal-containing complex or its solution and an oxidizer such as, for example, ozone, oxygen plasma or water plasma are employed. The gas lines connecting from the precursor canisters to the reaction chamber are heated to about 190° C. and the container of the Group 2 metal-containing complex is kept at about 190° C. for bubbling whereas the solution is injected into a vaporizer kept at 180° C. for direct liquid injection. 100 sccm of argon gas is preferably employed as a carrier gas to help deliver the vapor of the Group 2 metal-containing complex to the reaction chamber during the precursor pulsing. The reaction chamber process pressure is about 1 Torr. In a typical ALD or CCVD process, the substrate such as silicon oxide or metal nitride are heated on a heater stage in a reaction chamber that is exposed to the Group 2 metal-containing complex initially to allow the complex to chemically adsorb onto the surface of the substrate. An inert gas such as argon gas purges away unabsorbed excess complex from the process chamber. After sufficient Ar purging, an oxygen source is introduced into reaction chamber to react with the absorbed surface followed by another inert gas purge to remove reaction by-products from the chamber. The process cycle can be repeated to achieve the desired film thickness.

The following example illustrates the preparation of the metal-containing complexes with tridentate β-ketoiminate ligands as well as their use as precursors in metal-containing film deposition processes.

### EXAMPLES

The following example illustrates the preparation of the metal-containing complexes with tridentate β-ketoiminate ligands as well as their use as precursors in metal-containing film deposition processes.

### Example 1

### Synthesis of 2,2-dimethyl-5-[(2-methoxyethyl)amino]hex-4-en-3-one

To a solution of 15.00 g (105.49 mmol) 2,2-dimethyl-3,5-hexanedione in 100 mL THF loaded with 18.00 g (126.58 mmol) sodium sulfate was added 9.51 g (126.58 mmol) 2-methoxyethylamine. The reaction mixture was heated at 50° C. for several days after which THF was evaporated from mixture under vacuum yielding a yellow oil. Vacuum transfer of the residual oil heating at 130° C. under 150 mTorr vacuum yielded 17.31 g of a light yellow solid. The yield was 82%. ¹H NMR (500 MHz, C₆D₆): δ=11.45 (s, 1H), 5.21 (s, 1H), 2.93 (s, 3H), 2.90 (t, 2H), 2.78 (q, 2H), 1.49 (s, 3H), 1.31 (s, 9H).

### Example 2

### Synthesis of 2,2-dimethyl-5-[(2-methoxy-1-methylethyl)amino]hex-4-en-3-one

A reaction flask loaded with 12.6 g of 2,2-dimethylhexan-3,5-dione with excess 1-methoxy-2-propylamine and excess anhydrous sodium sulfate in diethyl ether was stirred until the dione was no longer observed by GC-MS. A clear solution was obtained by filtration and the filtrand was washed with diethyl ether. The solvent and excess amine was removed from the combined diethyl ether solution via a rotary evaporator. 13.2 g of product was obtained by vacuum distillation at 600 mtorr, 96 C. ¹H NMR (500 MHz, C₆D₆): δ=11.54 (s, 1H), 5.20 (s, 1H), 3.31 (m, 1H), 2.93 (s, 3H), 2.84 (m, 2H), 1.60 (s, 3H), 1.30 (s, 9H), 0.87 (d, 3H)

### Example 3

### Synthesis of 4-[(2,2-dimethoxyethyl)amino]pent-3-en-2-one

21.0 g of aminoacetaldehyde dimethylacetal (0.2 moles) were dissolved into 100 ml of tetrahydrofuran to which 20.0 g of 2,4-pentanedione (0.2 moles) were added dropwise over 5 minutes. The resulting mixture was then stirred overnight after which time the solvent and water of condensation formed during the reaction was removed by vacuum distillation. The final product was then vacuum distilled as a clear liquid. Yield of MeC(O)CH₂C(NCH₂CH(OMe)₂)Me=25.0 g (72% of theoretical). ¹H NMR: (500 MHz, C₆D₆): δ=1.47 (s, 3H), δ=2.00 (d, 3H), δ=2.98 (t, 2H), δ=3.05 (s, 6H), δ=4.00 (t, 1H), δ=4.89 (s, 1H), a=11.2 (bs, 1H); GCMS parent ion at 211 mu.

### Example 4

### Synthesis of 2,2-dimethyl-5-[(2,2-dimethoxyethyl)amino]hex-4-en-3-one

To a solution of 6.18 g (43.46 mmol) 2,2-dimethyl-3,5-hexanedione in 150 mL toluene was added 5.48 g (52.15 mmol) aminoacetalhedyde dimethylacetal followed by 12.0 g (84.48 mmol) of sodium sulfate. The reaction was heated at reflux for several days after which toluene was distilled off heating at 142° C. under static conditions. The resulting residual oil was heated to 75° C. under 100 mTorr vacuum for one hour to remove any residual toluene. Vacuum transfer of residual oil heating at 102° C. under 100 mTorr vacuum yielded a lime green oil weighing 7.17 g with a yield of 71%. ¹H NMR (500 MHz, C₆D₆): δ=11.43 (s, 1H), 5.21 (s, 1H), 4.02 (t, 1H), 3.03 (s, 6H), 2.99 (t, 2H), 1.50 (s, 3H), 1.29 (s, 9H).

### Example 5

### Synthesis of bis(2,2-dimethyl-5-[(2-methoxyethyl)amino]hex-4-en-3-onato)strontium

To a suspension of 0.94 g (4.56 mmol) Sr-isopropoxide in THF was added 2.00 g (10.04 mmol) 2,2-dimethyl-5-[(2-methoxyethyl)amino]hex-4-en-3-one in THF at room temperature. The reaction mixture turned to a solution in a couple of minutes which was stirred for 16 hours at room temperature. All volatiles were evaporated under vacuum to obtain a sticky yellow oil. After rinsing with cold (e.g., about −10° C.) hexane, 2.57 g of a sticky off-white solid was collected.

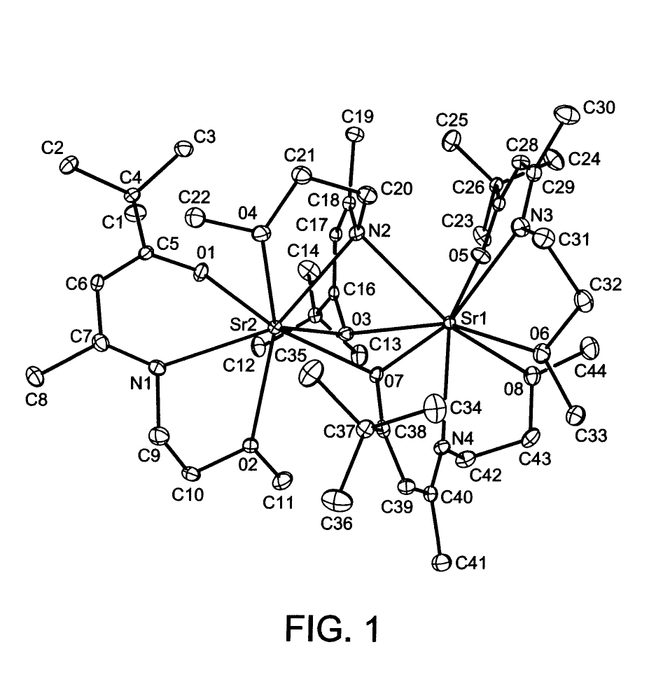

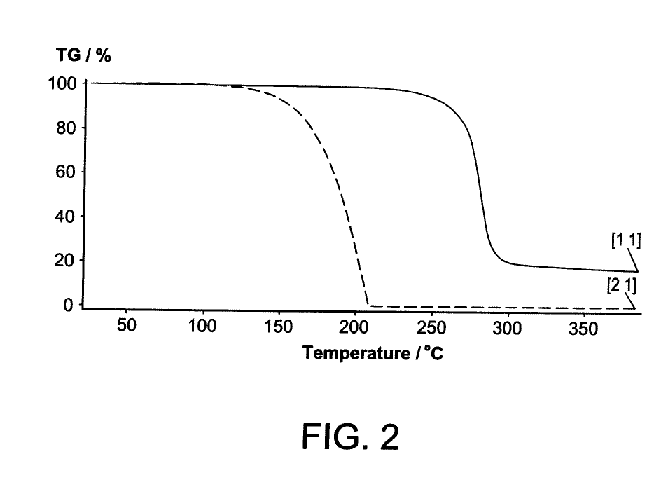

A single crystal of bis(2,2-dimethyl-5-[(2-methoxyethyl)amino]hex-4-en-3-onato)strontium was characterized by X-ray single crystal analysis, exhibiting that it is dimer in which one strontium atom is seven-coordinated with two nitrogen atoms and five oxygen atoms where the other eight-coordinated with three nitrogen atoms and five oxygen atoms. The structure of this compound is shown in FIG. 1. Referring to FIG. 2, the TGA of the complex indicates that it starts to vaporize at temperature greater than 250° C. There is about 15% residue, suggesting it is not a suitable precursor because it is not thermally stable. On the other hand, the pure ligand starts to vaporize at temperature greater than 120° C. and completely vaporize at temperature of 200° C.

### Example 6

### Synthesis of bis(2,2-dimethyl-5-[(2-methoxy-1-methylethyl)amino]hex-4-en-3-onato)strontium

To a suspension of 1.00 g (4.86 mmol) Sr-isopropoxide in 20 mL THF was added 1.95 g (9.72 mmol) 2,2-dimethyl-5-[(2-methoxy-1-methylethyl)amino]hex-4-en-3-one in 5 mL THF. The reaction mixture was left to stir at room temperature for 16 hours in which the suspension became a homogenous solution. THF was evaporated under vacuum yielding a waxy white solid. Removal of all volatile under vacuum transfer yielded about 1.70 g of off white solid. Recrystallization in octane gave colorless crystals with a yield of 72%. ¹H NMR (500 MHz, C₆D₆): δ=5.10 (s, 1H), 3.34 (d, 1H), 3.05 (s, 3H), 2.75 (d, 1H), 1.79 (s, 3H), 1.42 (s, 9H), 1.02 (d, 3H).

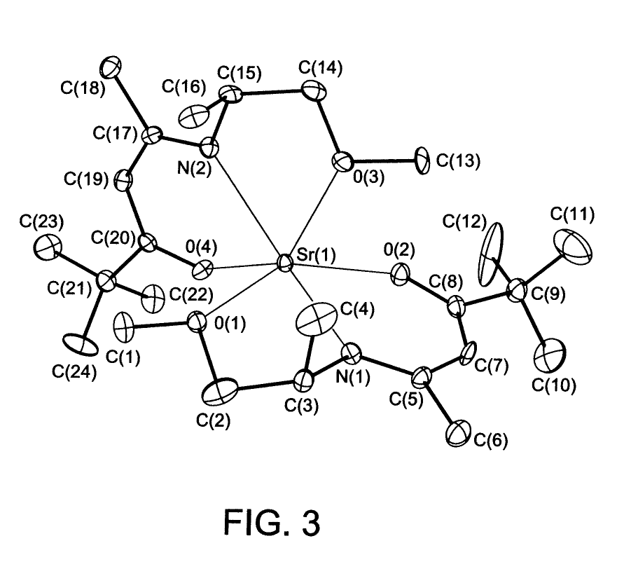

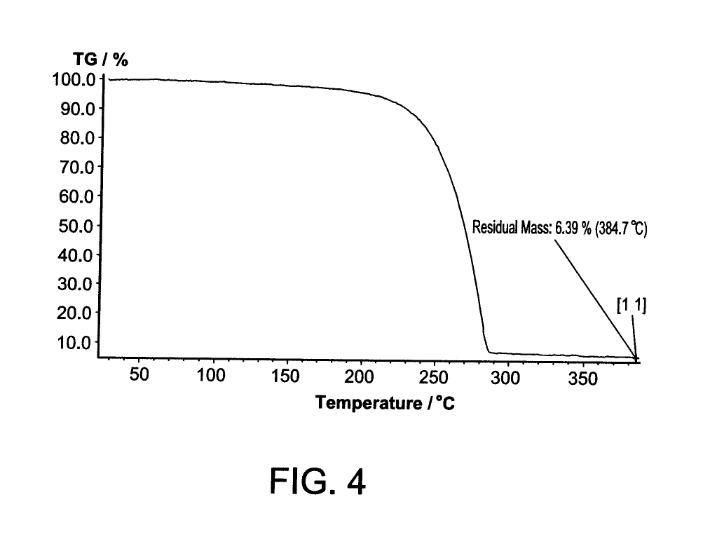

A single crystal of bis(2,2-dimethyl-5-[(2-methoxy-1-methylethyl)amino]hex-4-en-3-onato)strontium was characterized by X-ray single crystal analysis, exhibiting that it is a monomer in which the strontium atom is six-coordinated with two 2,2-dimethyl-5-[(2-methoxy-1-methylethyl)amino]hex-4-en-3-onato ligands. The structure of this compound is shown in FIG. 3. Referring to FIG. 4, the TGA analysis indicates that it is volatile with less than 5% residue, implying it can be employed as precursors in a CVD or ALD process.

### Example 7

### Synthesis of bis(4-[(2,2-dimethoxyethyl)amino]pent-3-en-2-onato)strontium

To a suspension of 0.50 g (2.43 mmol) Sr-isopropoxide in 15 mL THF was added 0.90 g (4.86 mmol) 4-[(2,2-dimethoxyethyl)amino]pent-3-en-2-one in 5 mL THF. The resulting suspension turned to a solution in approximately 10 minutes. The solution was stirred for 16 hours after which THF was evaporated under vacuum to give rise to about 1.20 g of an amber oil. The proton NMR suggested there are some unreacted free ligand which makes the product oily. H NMR of solid crashed out from a hexanes solution of the oil indicated it is an oligomer.

### Example 8

### Synthesis of bis(2,2-dimethyl-5-[(2,2-dimethoxyethyl)amino]hex-4-en-3-onato)strontium

To a suspension of 1.00 g (4.86 mmol) Sr-isopropoxide in 25 mL THF was added 2.33 g (10.21 mmol) 2,2-dimethyl-5-[(2,2-dimethoxyethyl)amino]hex-4-en-3-one in 5 mL THF. The resulting suspension turned to a solution in approximately 2 minutes which was left to stir at room temperature for 16 hours after which THF was evaporated from reaction mixture under vacuum to afford about 0.95 g. ¹H NMR (500 MHz, C₆D₆): δ=5.20 (s,1H), 3.93 (t, 1H), 3.22 (d, 2H), 3.09 (s, 6H), 1.78 (s, 3H), 1.41 (s, 9H).

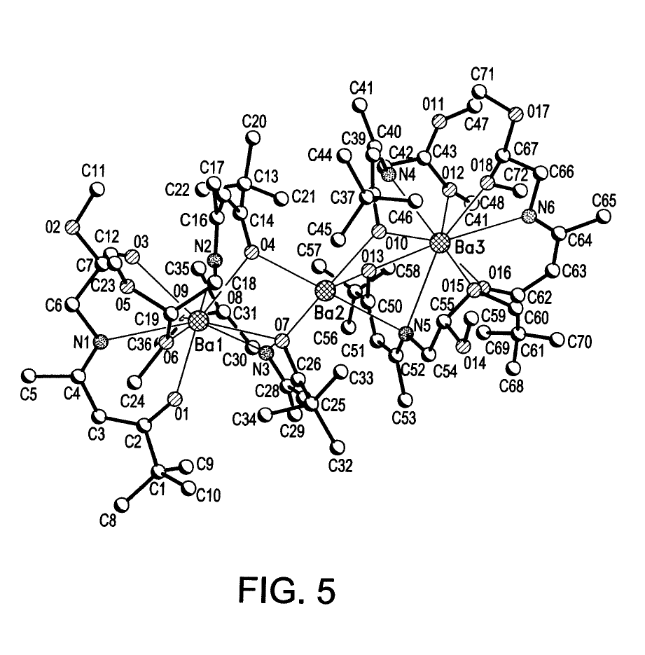

A single crystal of bis(2,2-dimethyl-5-[(2,2-dimethoxyethyl)amino]hex-4-en-3-onato)strontium was characterized by X-ray single crystal analysis, exhibiting that it is a one-dimensional polymer in which one strontium atom is eight-coordinated with two nitrogen atoms and six oxygen atoms. The structure of this compound is shown in FIG. 5.

### Example 9

### Synthesis of bis(2,2-dimethyl-5-[(2,2-dimethoxyethyl)amino]hex-4-en-3-onato)barium

To a clear solution of 0.96 g (2.09 mmol) Ba(N(SiMe3)2)2 in 20 mL THF was added 0.96 g (4.19 mmol) 2,2-dimethyl-5-[(2,2-dimethoxyethyl)amino]hex-4-en-3-one in 5 mL THF drop wise. Clear solution turned yellow and generated some evidence of heat and was stirred at room temperature for 3 hours. Removal of THF under vacuum provided a beige foam. 1.18 g of off-white solid was collected via washing with anhydrous hexanes. ¹H NMR (500 MHz, C₆D₆): δ=5.14 (s, 1H), 4.44 (b, 1H), 3.63 (b, 2H), 3.20 (s, 6H), 1.86 (s, 3H), 1.41 (s, 9H).

A single crystal of bis(2,2-dimethyl-5-[(2,2-dimethoxyethyl)amino]hex-4-en-3-onato)barrium was characterized by X-ray single crystal analysis, exhibiting that it is trimer polymer in which one barium atom is six-coordinated with two nitrogen atoms and four oxygen atoms whereas the other two barium atoms are nine-coordinated with three nitrogen atoms and six oxygen atoms. The structure of this compound is shown in FIG. 6.

### Example 10

### Synthesis of 2,2-dimethyl-5-[(tetrahydrofuran-2-ylmethyl)amino]hex-4-en-3-one

To a solution of 5.00 g (35.16 mmol) 2,2-dimethyl-3,5-hexanedione in 100 mL THF was added 6.00 g (42.24 mmol) sodium sulfate followed by 4.27 g (42.19 mmol) tetrahydrofurfurylmethylamine. The reaction was refluxed for 16 hours after which GC indicated reaction was complete. All volatiles were evaporated off under vacuum for several hours. 4.0 g of yellow oil was collected via short-path distillation. The yield was 50%.

### Example 11

### Synthesis of bis (2,2-dimethyl-5-[(tetrahydrofuran-2-ylmethyl)amino]hex-4-en-3-oneato)strontium

To a suspension of 1.00 g (4.86 mmol) Sr-isopropoxide in 20 mL THF was dropwise added 2.19 g (9.72 mmol) 2,2-dimethyl-5-[(tetrahydrofuran-2-ylmethyl)amino]hex-4-en-3-one in 5 mL THF to give rise to a suspension which turned into a solution in approximately 2 minutes. The reaction was stirred for 16 hours after which yellow solution was evaporated under vacuum to afford a foam weighing 2.83 g. ¹H NMR shows 50% conversion to product. ¹H NMR (500 MHz, C₆D₆): δ=5.13 (s, 1H), 3.88 (b, 2H), 3.65 (m, 1H), 3.34 (b, 1H), 3.09 (b, 1H) 1.83 (s, 3H), 1.50 (m, 2H), 1.43 (s, 9H), 1.41 (m, 1H), 1.32 (m, 1H).

### Example 12

A silicon wafer is maintained at a temperature of 250° C. in a reaction chamber at a pressure of 1.5 Torr. A Sr-containing compound of bis(2,2-dimethyl-5-[(2-methoxy-1-methylethyl)amino]hex-4-en-3-onato)strontium at 180° C. is introduced into the chamber for 5 seconds with 100 sccm N₂. A purge of 200 sccm N₂ follows for 10 seconds. A dose of ozone is then introduced for 10 seconds. This is followed by a 10 second purge with 200 sccm of N₂. This procedure is repeated for 200 cycles to produces a film of 6 nm thickness. EDX confirmed the resulting film contains strontium and oxygen.

### Comparative Example 1

In this experiment, the procedure outlined in Example 7 of JP06298714 was followed. This example should be compared to Example 5 above.

3.34 g (16.74 mmol) of 2,2-dimethyl-5-[(2-methoxyethyl)amino]hex-4-en-3-one in 10 mL THF was added dropwise to a suspension of 0.50 g (5.58 mmol) SrH₂ in 15 mL THF. A minor amount of bubbling was evident. The reaction mixture was stirred at room temperature for 16 hours, which gave rise to a grey slurry. THF was evaporated under vacuum to yield a waxy off-white solid. The TGA of the solid indicated that it was the unreacted 2,2-dimethyl-5-[(2-methoxyethyl)amino]hex-4-en-3-one. The crude waxy solid was then subjected to vacuum transfer similar to what is described in the JP06898714 reference. A white volatile solid was collected, which was 2,2-dimethyl-5-[(2-methoxyethyl)amino]hex-4-en-3-one as its TGA matched that of pure 2,2-dimethyl-5-[(2-methoxyethyl)amino]hex-4-en-3-one. The TGA diagram in JP06898712 is consistent with that of pure 2,2-dimethyl-5-[(2-methoxyethyl)amino]hex-4-en-3-one. Accordingly, it is evident that what is disclosed in prior art JP06298714 for the preparation of bis(2,2-dimethyl-5-[(2-methoxyethyl)amino]hex-4-en-3-onato)strontium was unsuccessful. Instead, the ligand itself was actually isolated.

### Comparative Example 2

In this experiment, the procedure outlined in paragraph 52 of JP06298714 was followed.

3.34 g (16.74 mmol) of 2,2-dimethyl-5-[(2-methoxyethyl)amino]hex-4-en-3-one in 5 mL THF was added dropwise to a suspension of 0.58 g (4.23 mmol) barium hydride in 20 mL THF. The resulting slurry was stirred for 16 hours after which THF was evaporated under vacuum yielding 3.10 g of a grainy liquid. Vacuum transfer at 150° C. under 125 mTorr vacuum provided approximately 2.00 g of a white solid that was characterized as 2,2-dimethyl-5-[(2-methoxyethyl)amino]hex-4-en-3-one, suggesting that the reaction did not occur.

TABLE 1
Summary of Examplary Complexes

| | | | |
|---|---|---|---|
| Example 5 | R¹ = Bu^{t}, R² = H, R³ = Me, | M = Sr | Structure A |
| | R⁴ = —CH₂CH₂, R⁵ = Me | | |
| Example 6 | R¹ = Bu^{t}, R² = H, R³ = Me, | M = Sr | Structure A |
| | R⁴ = —CH(Me)CH₂, R⁵ = Me | | |
| Example 7 | R¹ = Me, R² = H, R³ = Me, | M = Sr | Structure C |
| | R⁷ = —CH₂CH₂, R⁵ = R⁶ = Me | | |
| Example 8 | R¹ = Bu^{t}, R² = H, R³ = Me, | M = Sr | Structure C |
| | R⁷ = —CH₂CH₂, R⁵ = R⁶ = Me | | |
| Example 9 | R¹ = Bu^{t}, R² = H, R³ = Me, | M = Ba | Structure C |
| | R⁷ = —CH₂CH₂, R⁵ = R⁶ = Me | | |
| Example 11 | R¹ = Bu^{t}, R² = H, R³ = Me, | M = Sr | Structure B |
| | R⁴ = —CHCH₂, n = 3 | | |

The foregoing examples as summarized in Table 1 and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and scope of the invention, and all such variations are intended to be included within the scope of the following claims.

We claim:

1. A polydentate β-ketoiminate selected from the group consisting of: 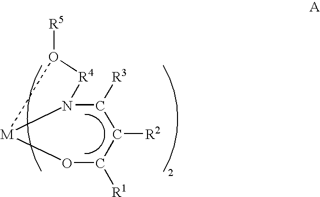
wherein
M is a group 2 metal selected from the group consisting of: magnesium, calcium, strontium, and barium;
R¹ is selected from the group consisting of: a C₁ to C₁₀ alkyl, a C₁ to C₁₀ alkoxyalkyl, a C₁ to C₁₀ alkoxy, a C₁ to C₁₀ fluoroalkyl, a C₃ to C₁₀ cycloaliphatic, and a C₆ to C₁₀ aryl;
R² is selected from the group consisting of: hydrogen, a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl;
R³ is selected from the group consisting of: a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl;
R⁴ is a C₁ to C₆ linear or branched alkylene; and
R⁵ is selected from the group consisting of: a C₁ to C₁₀ alkyl, a C₁ to C₁₀ fluoroalkyl, a C₃ to C₁₀ cycloaliphatic, and a C₆ to C₁₀ aryl, 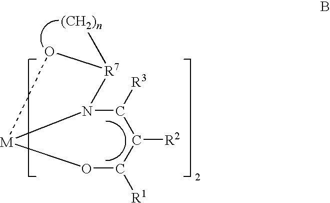
wherein
M is a group 2 metal selected from the group consisting of: magnesium, calcium, strontium, and barium;
R¹ is selected from the group consisting of: a C₁ to C₁₀ alkyl, a C₁ to C₁₀ alkoxyalkyl, a C₁ to C₁₀ alkoxy, a C₁ to C₁₀ fluoroalkyl, a C₃ to C₁₀ cycloaliphatic, and a C₆ to C₁₀ aryl;
R² is selected from the group consisting of: hydrogen, a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl;
R³ is selected from the group consisting of: a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl;
R⁷ is an organic group comprising 2 or 3 carbon atoms; and
n=3, 4, or 5, and 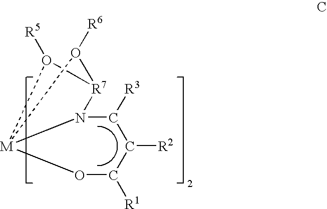
wherein
M is a group 2 metal selected from the group consisting of: magnesium, calcium, strontium, and barium;
R¹ is selected from the group consisting of: a C₁ to C₁₀ alkyl, a C₁ to C₁₀ alkoxyalkyl, a C₁ to C₁₀ alkoxy, a C₁ to C₁₀ fluoroalkyl, a C₃ to C₁₀ cycloaliphatic, and a C₆ to C₁₀ aryl;
R² is selected from the group consisting of: hydrogen, a C₁ to C₁₀ alkyl, a C₁ to C₁₀ alkoxyalkyl, a C₁ to C₁₀ alkoxy, a C₃ to C₁₀ cycloaliphatic, and a C₁ to C₁₀ aryl;
R³ is selected from the group consisting of: a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl;
R⁷ is an organic group comprising 2 or 3 carbon atoms; and
R⁵ and R⁶ are each independently selected from the group consisting of: a C₁ to C₁₀ alkyl, a C₁ to C₁₀ alkoxyalkyl, a C₁ to C₁₀ alkoxy, a C₁ to C₁₀ fluoroalkyl, a C₃ to C₁₀ cycloaliphatic, and a C₁ to C₁₀ aryl.

2. The polydentate β-ketoiminate of claim 1 wherein, for each of Structures A, B, and C, R¹ is a C₄ to C₆ bulky alkyl group.

3. The polydentate β-ketoiminate of claim 2 wherein the C₄ to C₆ alkyl group, R¹ is selected from the group consisting of: tert-butyl, iso-butyl, sec-butyl, and tert-pentyl.

4. The polydentate β-ketoiminate of claim 1 wherein, for Structure A, R⁴ is a linear alkylene bridge containing 2 or 3 carbon atoms

5. The polydentate β-ketoiminate of claim 1 wherein, for Structure A, R⁴ is a branched alkylene bridge containing 3 or 4 carbon atoms and having at least one chiral carbon atom; for Structure B, R⁷ contains 2 or 3 carbon atoms; and for Structure C, R⁷ is a branched alkylene bridge containing 3 or 4 carbon atoms and having at least one chiral carbon atom.

6. The polydentate β-ketoiminate of claim 1 wherein, for each of Structures A, B, and C, M is selected from the group consisting of: strontium and barium.

7. The polydentate β-ketoiminate of claim 1 wherein, for each of Structures A, B, and C, M is barium.

8. The polydentate β-ketoiminate of claim 1 wherein, for Structure B, R⁷ is

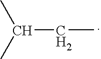

9. The polydentate β-ketoiminate of claim 1 wherein R⁷ is

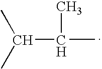

10. The polydentate β-ketoiminate of claim 1 wherein, for Structure C, R⁷ is

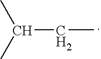

11. The polydentate β-ketoiminate of claim 1 selected from the group consisting of: bis(2,2-dimethyl-5-[(2-methoxyethyl)amino]hex-4-en-3-onato)strontium, bis(2,2-dimethyl-5-[(2-methoxy-1-methylethyl)amino]hex-4-en-3-onato)strontium, bis(,2-dimethyl-5-[(2-methoxy-1-methylethyl)amino]hex-4-en-3-onato)strontium, bis(2,2-dimethyl-5-[(2,2-dimethoxyethyl)amino]hex-4-en-3-onato)strontium, bis(2,2-dimethyl-5-[(2,2-dimethoxyethyl)amino]hex-4-en-3-onato)barium, and bis(2,2-dimethyl-5-[(tetrahydrofuran-2-ylmethyl)amino]hex-4-en-3-onato)strontium.

12. A liquid composition suitable for use to deposit a group 2 metal-containing film on a substrate by a chemical vapor deposition process, the composition comprising:
a polydentate β-ketoiminate according to claim 1; and
a solvent selected from the group consisting of: aliphatic hydrocarbons, aromatic hydrocarbons, ethers, esters, nitrites, organic esters; organic amines, polyamines, organic amides, alcohols, and mixtures thereof.

13. The composition of claim 12 wherein the solvent is a solvent selected from the group consisting of glyme solvents having from 1 to 20 ethoxy —(C₂H₄O)— repeat units; C₂-C₁₂ alkanols, organic ethers selected from the group consisting of dialkyl ethers comprising C₁-C₆ alkyl moieties, C₄-C₈ cyclic ethers; C₁₂-C₆₀ crown O₄-O₂₀ ethers wherein the prefixed Cᵢ range is the number i of carbon atoms in the ether compound and the suffixed Oᵢ range is the number i of oxygen atoms in the ether compound; C₆-C₁₂ aliphatic hydrocarbons; C₆-C₁₈ aromatic hydrocarbons; an organic amide of the form RCONR′R″ wherein R and R′ are alkyl having from 1-10 carbon atoms and they can be connected to form a cyclic group (CH₂)ₙ, wherein n is from 4-6, preferably 5, and R″ is selected from alkyl having from 1 to 4 carbon atoms and cycloalkyl.

14. The composition of claim 12 wherein the solvent is an organic amide selected from the group consisting of: N-methyl- or N-ethyl- or N-cyclohexyl-2-pyrrolidinones, N,N-diethylacetamide, and N,N-diethylformamide.

15. A vapor deposition process for forming a Group 2 metal oxide layer on a substrate, the process comprising the steps of:
dissolving a polydentate β-ketoiminate according to claim 1 in a solvent, wherein the solvent is selected from the group consisting of: aliphatic hydrocarbons, aromatic hydrocarbons, ethers, esters, nitrites, organic esters; organic amines, polyamines, organic amides, alcohols, and mixtures thereof; and
depositing a layer of the Group 2 metal-containing film on a substrate.

16. A vapor deposition process for forming a conformal metal oxide thin film on a substrate wherein a precursor source and an oxygen containing agent are introduced to a deposition chamber and a metal oxide film deposited on a substrate, the improvement which comprises using the metal containing complex of claim 1 as the precursor source.

17. The process of claim 15 wherein the vapor deposition process is selected from the group consisting of chemical vapor deposition and atomic layer deposition.

18. The process of claim 15 wherein the oxygen containing agent is selected from the group consisting of water, O₂, H₂O₂, ozone, water plasma, oxygen plasma, and mixtures thereof.

19. A polydentate β-ketoiminate selected from the group consisting of: 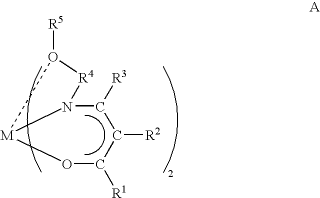
wherein
M is a group 2 metal selected from the group consisting of: magnesium, calcium, strontium, and barium;
R¹ is selected from the group consisting of: a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl;
R² is selected from the group consisting of: hydrogen, a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl;
R³ is selected from the group consisting of: a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl;
R⁴is a C₁ to C₆ linear or branched alkylene; and
R⁵ is selected from the group consisting of: a C₁ to C₁₀ alkyl, a C₃ to C₁₀ cycloaliphatic, and a C₆ to C₁₀ aryl, 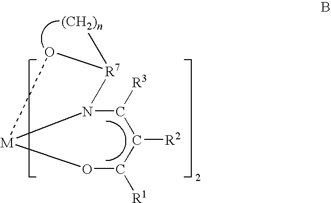
wherein
M is a group 2 metal selected from the group consisting of: magnesium, calcium, strontium, and barium;
R¹ is selected from the group consisting of: a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl;
R² is selected from the group consisting of: hydrogen, a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl;
R³ is selected from the group consisting of: a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl;
R⁷ is an organic group comprising 2 or 3 carbon atoms; and
n=3, 4, or 5, and 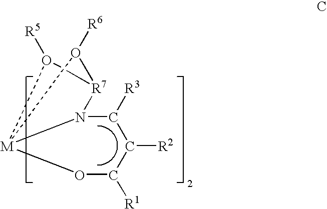
wherein
M is a group 2 metal selected from the group consisting of: magnesium, calcium, strontium, and barium;
R¹ is selected from the group consisting of: a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl;
R² is selected from the group consisting of: hydrogen, a C₁ to C₁₀ alkyl, a C₁ to C₁₀ alkoxyalkyl, a C₁ to C₁₀ alkoxy, a C₃ to C₁₀ cycloaliphatic, and a C₁ to C₁₀ aryl;
R³ is selected from the group consisting of: a C₁ to C₁₀ alkyl, a C₁ to C₁₀ alkoxyalkyl, a C₁ to C₁₀ alkoxy, a C₃ to C₁₀ cycloaliphatic, and a C₆ to C₁₀ aryl;
R⁷ is an organic group comprising 2 or 3 carbon atoms; and
R⁵ and R⁶ are each independently selected from the group consisting of: a C₁ to C₁₀ alkyl, a C₁ to C₁₀ alkoxyalkyl, a C₁ to C₁₀ alkoxy, a C₃ to C₁₀ cycloaliphatic, and a C₁ to C₁₀ aryl.

20. A method for making a metal-containing polydentate β-ketoiminate comprising reacting a polydentate β-ketoiminate ligand with a metal alkoxide in an organic solvent or a mixture of solvents.

21. The method of claim 20 wherein the metal alkoxide comprises an alkoxide selected form the group consisting of: methoxide, ethoxide, iso-propoxide, n-propoxide, tert-butoxide, sec-butoxide, iso-butoxide, and mixtures thereof.

22. The method of claim 21 wherein the metal alkoxide is strontium iso-propoxide.

23. The method of claim 20 wherein the organic solvent is selected from the group consisting of: THF, toluene, hexanes, ether, and mixtures thereof.

24. The method of claim 20 wherein the metal-containing polydentate β-ketoiminate is selected from the group consisting of: 2,2-dimethyl-5-[(2-methoxyethyl)amino]hex-4-en-3-one, 2,2-dimethyl-5-[(2-methoxy-1-methylethyl)amino]hex-4-en-3-one, 4-[(2,2-dimethoxyethyl)amino]pent-3-en-2-one, 2,2-dimethyl-5-[(2,2-dimethoxyethyl)amino]hex-4-en-3-one, and 2,2-dimethyl-5-[(tetrahydrofuran-2-ylmethyl)amino]hex-4-en-3-one.
